(19) United States
(12) Patent Application Publication
(10) Pub. No.: US 2021/0346109 A1
(43) Pub. Date: Nov. 11, 2021

(54) MONITORING PERFORMANCE DURING MANIPULATION OF USER INPUT CONTROL DEVICE OF ROBOTIC SYSTEM

(71) Applicant: CMR SURGICAL LIMITED, Cambridge, Cambridgeshire (GB)

(72) Inventors: Luke David Ronald Hares, Cambridge, Cambridgeshire (GB); Paul Christopher Roberts, Cambridge, Cambridgeshire (GB)

(21) Appl. No.: 17/282,655

(22) PCT Filed: Oct. 3, 2019
(86) PCT No.: PCT/GB2019/052792

§ 371 Date: Apr. 2, 2021

(30) Foreign Application Priority Data
Oct. 3, 2018 (GB) 1816167.9

(51) Int. Cl. A61B 34/00; A61B 34/37; A61B 90/00; B25J 9/16; B25J 13/02

(52) U.S. Cl. CPC A61B 34/74; A61B 34/37; A61B 34/25; A61B 2090/0807; A61B 34/76; B25J 9/1689; B25J 13/025; A61B 90/08

(57) ABSTRACT

A surgical robotic system, comprising: a surgical robot; a user input device coupled to the surgical robot and manipulatable by a user to control operation of the surgical robot, the user input device comprising one or more sensors configured to collect data as the user manipulates the user input device; a processor unit configured to: analyse the collected data to determine whether a parameter associated with the operation by the user of the surgical robot has a desired working value; and generate an output signal indicating responsive action is to be taken in response to determining from the collected data that the parameter does not have a desired working value.

### FIELD

This invention relates to monitoring performance during user-controlled manipulation of an input control device of a robotic system through the collection of data using one or more sensors on the input control device.

### BACKGROUND

Surgical robots are used to perform medical procedures on humans and/or animals. A surgical robot typically comprises a moveable mechanism (robot arm) which supports an end effector which is a surgical instrument. The mechanism can be reconfigured to move the end effector to a surgical site and to operate the end effector to perform surgery. The robot is typically controlled by a user (e.g. a surgeon) operating a console which is communicatively coupled to the robot. The console may comprise one or more user input devices (e.g. a controller) coupled to the surgical robot by data links. A user can control movement of the end effector by suitable manipulation of the user input device. For example, the user may move the user input device in three-dimensional space to effect corresponding movement of the end effector.

One potentially convenient aspect of robotic surgery compared to manual surgery is that it permits data to be gathered more easily during the performance of a surgical procedure. It would be desirable to leverage the ability to collect data to improve the safety and/or efficacy of procedures performed by surgical robots.

### SUMMARY

According to the present invention there is provided a surgical robotic system as set out in the appended claims.

### BRIEF DESCRIPTION OF DRAWINGS

The present invention will now be described by way of example with reference to the accompanying drawings. In the drawings:

FIG. 1 shows a surgical robotic system;

FIG. 2 shows a control system of the surgical robotic system;

FIG. 3 shows an example user input device for controlling movement of a robotic arm of the surgical robotic system.

### DETAILED DESCRIPTION

The present disclosure is directed to a robotic system comprising a robot arm and a user input device manipulatable by a user to control operation of the robot arm. The user input device forms part of a console the user stands at, or mans, during use to perform a surgical procedure. The console comprises one or more sensory devices for capturing data pertaining to the user during use of the surgical robotic system, e.g. as the user manipulates the user input device. The data pertaining to the user may be data characterising the state of the user in some way, e.g. their physiological state, or it may be data associated with a physiological or biometric parameter. In one set of examples, the sensory devices do not form part of the user input device but could be, for example, an image capture device (e.g. a camera) for capturing images of the user during use of the robotic system, or an audio capture device (e.g. a microphone) for capturing audio data for the user.

In another set of examples, the sensory devices do form part of the user input device, and the user input device comprises a set of one or more sensors for collecting data as the user manipulates the device to control operation of the robot arm. The data might include physiological or biometric data of the user (e.g. blood pressure, body temperature, perspiration rate etc.) and/or data characterising the manipulation of the user input device by the user such as, for example, the orientation of the user input device, the range of motion through which the device is positioned by the user; the force applied by the user to the user input device etc.

The collected data is analysed by a processor unit to determine whether a parameter associated with the user's operation of the surgical robot has a desired working value. The desired working value might for example be a predetermined value. The desired working value might represent a safe working value. A desired working value might be a value located within some desired working range. The desired working value might be a value for a physiological and/or biometric parameter of the user, or it might be a value of a parameter characterising the manipulation of the user input device by the user. The desired working value (and the desired working range, if appropriate) might be stored in a memory accessible by the processor unit.

If it is determined that the parameter does not have a desired working value, the processor unit generates and outputs a feedback signal indicating responsive action is to be taken. The feedback signal might be output to a further component of the robotic system. The feedback signal might directly cause the further component of the robotic system to take a responsive action, or it might cause the further component of the robotic system to provide feedback to the user (e.g. audio, visual and/or haptic) to indicate that responsive action by the user is required. Examples of types of feedback signal and the associated responsive actions will be provided below. By collecting data as the user manipulates the user input device and generating an output signal if the data indicates a parameter associated with the user's operation of the surgical robot does not have a desired value, responsive action can be effected (directly or indirectly), thereby increasing the safety and/or efficacy of the surgical procedure.

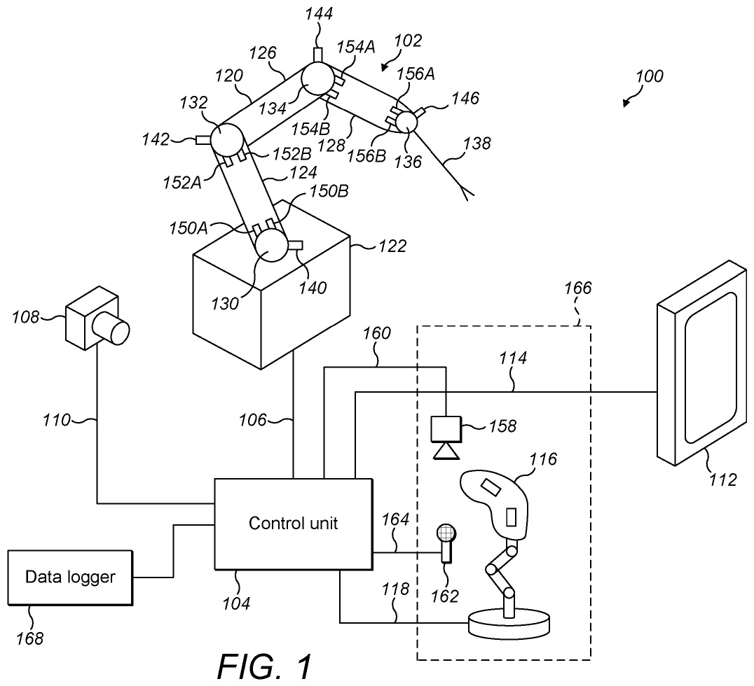

FIG. 1 shows an example of a surgical robotic system, denoted generally at 100. The robotic system comprises surgical robot 102 coupled to a control unit 104 by a data link 106. The system further comprises a user console, or user station, denoted generally at 166. The console 166 comprises a user input device 116, an image capture device 158 (e.g. a camera), and an audio capture device 162 (e.g. a microphone).

The control unit 104 is coupled to an audio output device 108 by data link 110; a visual display device 112 by data link 114 the user input device 116 by data link 118; the image capture device 158 by data link 160 and the audio capture device by data link 164. Each of the data links may be wired communication links. Each of the data links may be wireless communication links. The data links may be a mixture of wired and wireless communication links. That is, one or more of data links 106, 110, 114 and 118 may be wired communication links and one or more may be wireless communication links. In other examples, any of the data links may be a combination of a wired and wireless communication link.

The audio output device 108 is configured to output audio signals. Audio output device 108 may be a speaker. Visual display device 112 is configured to display images. The images may be static images or moving images. Visual display device might for example be a screen, or monitor.

The control unit 104 may be located locally to the surgical robot 102 (e.g. within the same room, or operating theatre), or it may be located remotely of it. Similarly, the user input device 116 may be located locally or remotely of the surgical robot 102. The audio output device 108 and visual display device 112 may be located locally to the user input device 116. The devices 108 and 112 may be located in relative proximity to the user input device 116 so that outputs from these devices (audio and visual signals respectively) are capable of being detected by a user operating the surgical robot 102. The image capture device 158 and audio capture device 162 form part of console 166 and so are located locally to user input device 116 so that image capture device 158 can capture visual images of a user operating the surgical robot 102 and audio capture device 162 can capture sounds emitted from the user operating the surgical robot 102. This will be explained in more detail below.

The robot 102 comprises a robotic arm 120, which in this example is mounted to base 122. The base 122 may in turn be floor mounted, ceiling mounted, or mounted to a moveable cart or an operating table. The robot arm 120 terminates in an end effector 138. The end effector 138 might be, for example, a surgical instrument of endoscope. A surgical instrument is a tool for performing some operational function, for example cutting, clasping, irradiating or imaging.

The robot arm 120 comprises a series of rigid portions, or links (124, 126, 128) interconnected by successive joints 132 and 134. That is, each successive pair of links is interconnected by a respective joint; i.e. the links are articulated with respect to each other by a series of joints. The robot further comprises a joint 130 interconnecting the most proximal link 124 with base 122, and joint 136 interconnecting the most distal link 128 of the robot arm with instrument 138. The joints 130-136 may comprise one or more revolute joints that each permit rotation about a single axis. The joints 130-136 may comprise one or more universal joints that each permit rotation about two orthogonal axes.

Though the robot arm 120 is shown comprising a series of three rigid links, it will be appreciated that arm here is merely exemplary and that in other examples the arm may include a greater or fewer number of links, where each successive pair of links in the series is interconnected by a respective joint, the proximal link is connected to a base via a joint, and the terminal link is connected to an end effector via a joint.

The surgical robot arm 120 further comprises a set of actuators 140, 142, 144 and 146 for driving motion about joints 130, 132, 134 and 136 respectively. That is, motion about each joint of the robot arm can be driven by a respective actuator. The operation of the actuators (e.g. the driving and braking of each actuator) may be controlled by signals communicated from the control unit 104. The actuators might be motors, e.g. electric motors.

The robot arm 120 also includes a plurality of sets of sensors. In this example, the robot arm 120 includes a set of sensors for each joint, denoted 150_{A,B}, 152_{A,B}, 154_{A,B} and 156_{A,B}. In this example, the set of sensors for each joint includes a torque sensor (denoted by the suffix ‘A’) and a position sensor, or position encoder (denoted by the suffix ‘B’). Each torque sensor 150-156_{A} is configured to measure the torque applied at a respective joint, i.e. for measuring the torque applied about the joint's rotation axis. The measured torque might include internally applied torque at the joint provided by the respective actuator driving that joint and/or externally applied torque at the joint, e.g. from the weight of the robot arm or a manual force applied by a user. Each position sensor 150-156_{B} measures the positional configuration of a respective joint. The sensors 150-156_{A,B} may output signals over data link 106 containing sensed data indicating measured torque values and positional configurations of the joints to the control unit 104.

The user input device 116 enables a user to operate the surgical robot 102. The user manipulates the user input device 116 to control the position and movement of the robot arm. The user input device 116 outputs user-control signals to the control unit 104 over data link 118 containing data indicative of a desired configuration of the robot arm 120. The control unit 104 can then output drive signals to the actuators 140-146 of the robot arm 120 to effect a desired motion about the robot arm joints 130-136 in dependence on the signals received from the user input device 116 and from the robot arm sensors 150-156_{A,B}.

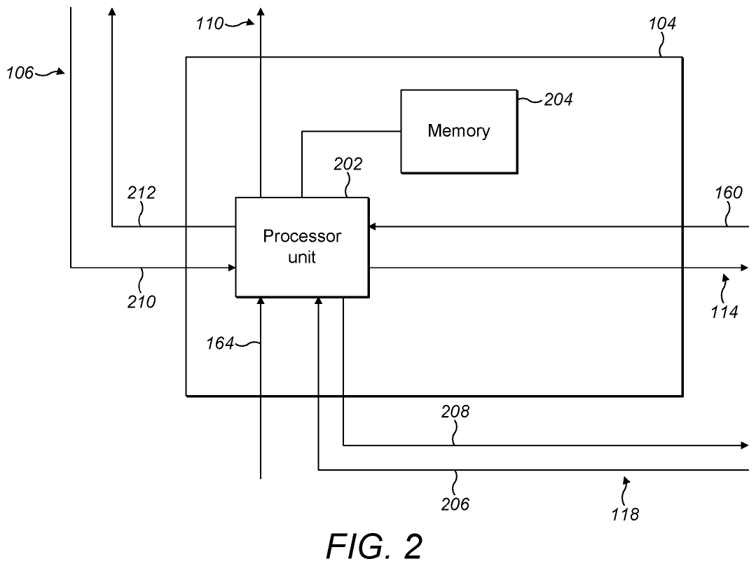

An exemplary structure of the control unit 104 is shown in FIG. 2. The control unit comprises a processor unit 202 and a memory 204. The processor unit 202 is coupled to the memory 204.

The processor unit 202 receives user-control signals from the input device 116 over communication path 206 indicating a desired configuration of the robot arm 120. The communication path 206 forms part of the data link 118. Communication path 208 from the processor unit 202 to the user input device also forms part of data link 118 and permits signals to be communicated from the processor unit 202 to the user input device 116, which will be explained in more detail below.

The processor unit 202 also receives signals containing sensed data from the sensors 150-156_{A,B} of the robot arm 120 over communication path 210, which forms part of the data link 106. The processor unit 202 communicates motion-control signals to the actuators of the robot arm 120 over communication path 212 to effect a desired motion about the joints 130-136. The motion-control signals may include drive signals to drive motion about a joint and/or brake signals to brake an actuator to arrest motion about a joint. Communication path 212 also forms part of the data link 106. The processor unit 202 may communicate the motion-control signals to the actuators of the robot arm 120 in dependence on the motion control signals received from the user input device 116 and the signals containing sensed data received from the sensors 150-156_{A,B}.

As shown, the processor unit 202 generates signals for communication to the audio output device 108 over data link 110, signals for communication to the visual display device 112 over data link 114 and signals for communication to the user input device 116 over communication path 208 of data link 118. The generation of these signals will be explained in more detail below.

The memory 204 is an example of a storage medium and may store in a non-transitory way computer-readable code that can be executed by the processor unit 202 to perform the processes described herein. For example, on executing the code, the processor unit 202 determines the motion-control signals for communication over data link 106 to the actuators of the robot arm 120 in dependence on the signals received from the user input device 116 and the signals received from the robot arm's sensors 150-156_{A,B}. Processor unit 202 may also execute code stored in non-transitory form in memory 204 to generate the signals communicated over data link 110 to audio output device 108, the signals communicated over data link 114 to the visual display device 112 and the signals communicated over communication path 208 of data link 118 to the user input device 116.

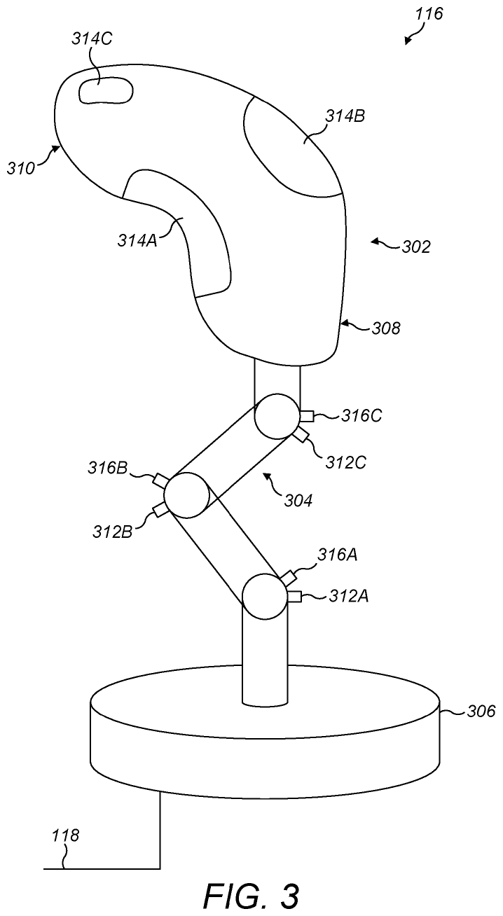

FIG. 3 shows a more detailed view of an exemplary user input device 116. In this example, the user input device comprises a controller 302 supported by an articulated linkage 304.

The articulated linkage is connected to a platform, or base, 306. The linkage 304 permits the controller 302 to be manoeuvred in space with a number of degrees of freedom. The degrees of freedom may include at least one translational degree of freedom and/or one rotational degree of freedom. The number of degrees of freedom may vary depending on the arrangement of the linkage, but in some examples the linkage 304 may permit the controller to be manoeuvred with six degrees of freedom (three translational degrees of freedom and three rotational degrees of freedom). The articulated linkage 304 may comprise plurality of rigid links interconnected by joints. The links may be rigid. Each successive pair of links may be interconnected by a respective joint. The links and their interconnected can provide the translational degrees of freedom of the controller 302. The linkage may further comprise a gimbal (not shown in FIG. 3) for providing the rotational degrees of freedom (e.g. enabling the controller to be moved in pitch and/or roll and/or yaw). Alternatively, the angular degrees of freedom may be provided by the joints of the linkage, for example one or more of the linkage joints may be spherical joints.

The controller 302 is designed to be held in the user's hand. A user can manipulate the controller in three-dimensional space (e.g. by translation and/or rotation of the controller) to generate user control signals communicated to the control unit 104. The controller comprises a grip portion 308 and a head portion 310. When in correct use, the grip portion 308 sits in the palm of the user's hand. One or more of the user's index fingers wrap around the grip portion. When in correct use, the user's hands do not come into contact with the head portion 310 of the controller. The grip portion 308 in this example forms a first terminal portion of controller 302, and the head portion 310 forms a second terminal portion of controller 302. The first terminal portion might be referred to as a proximal terminal portion and the second terminal portion might be referred to as a distal terminal portion.

The grip portion 308 may be of any convenient shape: for example of generally cylindrical form. It may have a circular, elliptical, square or irregular cross-section. The grip could be configured to be gripped by one, two or three fingers. The grip portion may be slimmer than the head portion. In cross-section perpendicular to the extent of the grip portion, the grip portion may be generally circular.

The head portion 310 is rigidly attached to the grip portion 308. The grip and head portion may be parts of a common housing of the controller 302.

The controller may additionally comprise one or more user interface inputs, such as buttons, triggers etc (omitted from FIG. 3 for clarity). The user interface inputs may be used to enable the user to provide a functional input to the surgical robot, e.g. controlling operation of the surgical instrument.

In this example, the user input device 116 generates the user control signals indicating a desired position and orientation of the end effector 138 in dependence on the configuration of the articulated linkage 304. The configuration of the linkage 304 can be used to calculate the position and orientation of the hand controller 302. The configuration of the linkage 304 can be detected by sensors 312_{A,B,C} on the linkage. That is, the input-device sensors 312_{A,B,C} may operate to sense the configuration of each link of the linkage 304. For example, each of sensors 312_{A,B,C} may measure the positional configuration of a respective joint of the articulated linkage 304, i.e. each of sensors 312_{A,B,C} might be position sensors that measure the position of a respective joint of the linkage 304. The sensed data from sensors 312_{A,B,C} is then used to calculate the position of the hand controller 302. If the linkage includes a gimbal, the user input device 116 may further include sensors for sensing the angular position of the gimbal. The sensed data from the gimbal sensors can be used to calculate the orientation of the controller 302. These calculations may be performed by the user input device 116, for example by a processor housed within the user input device. Alternatively, the calculations of the controller's position and/or orientation may be performed by the processor unit 202 from the joint and/or gimbal positions of linkage sensed by the sensors. In general, the user input device 116 can output a user control signal indicating the position and orientation of the hand controller 302 to the control unit 104 over data link 118. Those control signals may contain position and orientation data for the controller 302 (if the position and orientation of the controller 302 is calculated by the user input device 116), or they may contain joint and optionally gimbal position data for the linkage 304 (if the position and orientation is calculated by the processor unit 202). The control unit 104 receives the user control signals and calculates from those signals a desired position and orientation of the end effector 138. That is, the control unit 104 may calculate a desired position and orientation of the end effector 138 from the position and orientation of the hand controller 302. Having calculated the desired position and orientation of the end effector 138, the control unit 104 calculates the configuration of the arm 120 to achieve that desired position and orientation.

Thus, in summary, when in use, a user manipulates the user input device 116 by manoeuvring the controller 302 in space causing movement of the articulated linkage 304. The configuration of the linkage 304 can be sensed by the linkage sensors and used to calculate a position and orientation of the hand controller 302, with a user-control signal containing data indicating that position and orientation (and hence indicating the desired position and orientation of the end effector 138) being communicated from the user input device 116 to the control unit 104.

Though only a single hand controller 302 is shown in FIG. 3, it will be appreciated that in some examples the user input device 116 may comprise two hand controllers. Each hand controller may adopt the form of controller 302 described above. Each hand controller might be supported by a respective linkage. Each hand controller may be configured to generate control signals to control a respective end effector, e.g. a surgical tool and an endoscope. The end effectors may be located on a single robotic arm or on respective arms. In other examples, each controller may be configured to control a single end effector.

In accordance with the examples described herein, the user input device 116 comprises a set of sensors that are configured to collect data as the user manipulates the device 116, where that data is associated with the operation by the user of the surgical robot 102. The collected data is communicated to the processor unit 202, where it is analysed to determine whether a parameter associated with the operation by the user of the surgical robot has a desired working value. If the processor unit determines that the parameter does not have a desired value, the processor unit 202 generates an output signal indicating responsive action is to be taken. The output signal generated by the processor unit 202 might be a feedback signal in the sense it indicates an action is to be taken. The output signal might indicate that responsive action is to be taken by a component of the robotic system 100 or by the user of the robotic system 100. Various examples of the types of sensors and feedback signals will now be described.

The set of sensors that measure the data to be analysed by the processor unit 202 may include the input device sensors 312_{A,B,C}. In other examples, the set of sensors that measure the data to be analysed by the processor unit 202 might be, or might include, further sensors in addition to the input device sensors 312_{A,B,C}. An example set of such sensors are shown in FIG. 3 at 314_{A,B,C}.

Sensors 314 may comprise one or more sensors configured to collect physiological data for the user of the device 116. In this example, those sensors are sensors 314_{A,B}. The physiological data sensors 314_{A,B} may be arranged to collect physiological data from the user's hands during operation of the device 116. To aid such data collection, the physiological data sensors may be positioned on the device 116 to be in contact with the user's hand as the user operates the input device 116. That is, the sensors may be positioned on the input device 116 to be in contact with the user's hand during normal use of the user input device 116 to control operation of the surgical robot 102. ‘Normal use’ may refer to the case when the user's hand is in an intended, or desired position on the input device 116. The intended, or desired, position may be an ergonomic position. It will be appreciated that the position of the user's hand in normal use will depend on the shape and configuration of the user input device.

In the example shown in FIG. 3, sensors 314_{A,B} are positioned on the controller 302 that is grasped by the user's hand during use. In particular, the sensors 314_{A,B} are positioned on the grip portion 308 of the controller 302 so that they are in contact with the user's hand when the user grips the controller 302. In the example arrangement shown, sensor 314_{A} is positioned to be located under the user's fingers when the user grips the controller, and sensor 314_{B} is positioned to be located under the palm, or the base of the user's thumb. Locating the sensors to be positioned under the user's fingers may conveniently enable physiological data to be collected from multiple different locations on the user's hand simultaneously. This may enable the veracity of any conclusions drawn from an analysis of the data by the processor 202 to be improved (e.g. by reducing the incidence of false positives) and/or improve the rate of data collection during use of the input device 116. It will be appreciated that in other examples the sensors may be located at different positions on the controller.

Conveniently, the sensors 314_{A,B} may be located at the surface of the controller 302 to facilitate good contact with the user's hand.

Types of physiological data for the user that might be collected by the sensors 314_{A,B} might include, for example, skin temperature data; pulse rate data; blood oxygen saturation level data; perspiration rate data; ionic concentration in perspiration data; hydration level data and blood pressure data. Skin temperature data might be measured by a temperature sensor. The user's pulse rate data might be measured by a photoplethysmography (PPG) sensor or an electrocardiography (ECG) sensor. In the case of ECG, ECG sensors may be provided on both hand controllers of the user input device 116. The blood oxygen saturation level data might be measured by a PPG sensor or a pulse oximetry sensor. Perspiration rate data might be measured by a perspiration rate sensor, which might be for example a skin conductance sensor or a sweat-rate sensor. The skin conductance sensor may comprise one or more electrodes configured to measure conductance, which is dependent on electrolyte levels contained in perspiration. The sweat-rate sensor might comprise a humidity chamber for collecting moisture evaporated from the skin, and one or more humidity sensors located within the chamber to measure the humidity level within the chamber. Ionic concentration data might be measured by an ionic concentration sensor. The ionic concentration sensor might comprise one or more electrodes for measuring skin conductivity, which is indicative of ionic concentration levels (the higher the concentration level, the higher the conductivity). Hydration level data may be collected by a hydration sensor. The hydration sensor may for example measure one or more of: skin elasticity, blood glucose concentration (through light-based detection), perspiration conductivity, or skin pH.

Each of sensors 314_{A} and 314_{B} may collect a different type of physiological data. That is, sensor 314_{A} may collect a first type of physiological data and sensor 314_{B} may collect a second type of physiological data. In other examples, each of sensors 314_{A,B} may collect the same type of physiological data (e.g. both sensors may collect temperature data, for example).

Though only two sensors for collecting physiological data are shown in the example illustrated in FIG. 3, it will be appreciated that the user input device 116 may include any suitable number of sensors for collecting physiological data of the user. The user input device may for example include three, four, five or more sensors for collecting physiological data. In general, the user input device 116 may include a set of one or more sensors for collecting physiological data for the user. The user input device may include a plurality of sensors for collecting physiological data for the user. The plurality of sensors may collect one or more different types of physiological data. Thus, in one example, the user input device comprises a plurality of sensors each configured to collect physiological data of the same type; in another example, the plurality of sensors collect a plurality of types of physiological data, for instance each of the plurality of sensors may collect a different respective type of physiological data.

Data collected by the sensors 314_{A,B} is communicated to the processor unit 202 over data path 206 of communication link 118. The collected data may be streamed to the processor unit 202. Alternatively, the collected data may be communicated to the processor unit 202 in bursts.

The processor unit 202 operates to analyse the collected data received from the user input device 116 to determine whether a parameter associated with the user's operation of the surgical robot has a desired working value. Continuing the present example, in which the collected data is physiological data, the parameter associated with the user's operation of the surgical robot is a physiological parameter of the user during the user's operation of the surgical robot. The physiological parameter might be, for example (depending on the data collected): the user's temperature; user's pulse rate; user's blood oxygen saturation level; user's perspiration rate; user's ionic concentration; user's hydration level etc.

The processor unit 202 may determine the value of a physiological parameter from the collected data (e.g. pulse rate, user temperature, user hydration level, perspiration rate etc.) and determine whether the value for that physiological parameter is a desired value. The processor unit 202 might analyse the collected data to determine a time-averaged value of the physiological parameter over a period of time, and determine whether that time-averaged value is a desired value. For example, the collected data from sensors 314_{A,B} may specify values of the physiological parameters and a timestamp associated with each value. These values may be averaged over a period of time to calculate an average physiological parameter value for that period of time.

The desired value may be some specified value. It may be a predetermined value. The desired working value of the parameter may be any value within a specified range, or a value above or below a specified threshold. The desired working value may be a value indicative of a good, or acceptable, physiological state. The desired working value might be a ‘safe’, or normal value, e.g. a clinically acceptable value.

Desired values, ranges of values and/or threshold values for the physiological parameters may be stored in the memory 204 of the control unit 104. The processor unit 202 may access the values stored in the memory 204 to determine whether a physiological parameter has a desired working value, e.g. by comparing a value of the physiological parameter determined from the collected data with the desired values, value ranges or thresholds stored in the memory 204.

If a physiological parameter does not have a desired working value, this might indicate that the user is not in an optimal or desired state to control operation of the surgical robot 102. For example, hydration levels are known to affect mental performance and concentration levels. If the user's hydration levels as determined from data collected from sensors 314_{A,B} are not at a desired level (e.g. they are below a threshold), this may indicate the user's concentration or mental capacity to control the surgical robot is impaired. Other physiological parameters might serve as biomarkers for an impaired ability of the user to control the surgical robot. For example, a pulse rate above a specified value might indicate that the user is under excessive levels of stress, or nervousness. A perspiration rate that exceeds a specified value may similarly indicate excessive stress or anxiety levels. A skin temperature above a specified threshold might indicate that the user is unwell (e.g. suffering a fever), or physically over-exerted. An oxygen saturation rate that is below a threshold might indicate that the user is suffering from symptoms including headaches, confusion, lack of coordination or visual disorders. It will be appreciated that the physiological parameters might serve as biomarkers for other types of conditions.

Thus, in response to detecting that a physiological parameter does not have a desired value, the processor unit 202 generates and outputs a signal indicating responsive action is to be taken. This signal may be output to another component of the robotic system 100 to cause that component to perform a dedicated responsive action. Various examples of this will now be described.

In one example, the processor unit 202 outputs a control signal to brake the actuators 140-146. That is, the processor unit 202 outputs a braking signal to the actuators 140-146 in response to detecting that a physiological parameter does not have a desired value. Thus, the signal output by the processor unit 202 may arrest motion of the surgical robot 102 and lock each joint 130-136 of the robot arm. In other words, the signal output from the processor unit 202 may lock the position of the robot arm 120 in place.

In another example, the processor unit 202 outputs a control signal to suspend operation of the end effector 138. The control signal might lock the end effector. For example, if the end effector is a surgical instrument that includes a pair of grippers, the control signal may cause the grippers to be locked in place. If the surgical instrument includes cutting elements (e.g. blades), the control signal may cause the cutting elements to be locked in place. If the surgical instrument is a cauterising or irradiating tool, the control signal might terminate the power supply to the instrument.

In another example, the processor unit 202 outputs an alert signal to the audio output device 108 and/or the visual display device 112. The alert signal may cause the audio output device 108 to generate an audio signal, e.g. an audio alarm. The alert signal may cause the visual display device 112 to display a visual image, e.g. a warning image or visual alert. The audio signal and/or displayed visual image may serve to alert the user of input device 116 and/or other personal that the user's physiological parameters do not have a desired value. This may indicate that a change in user is required, or that the user requires a break from operating the surgical robot.

In the examples described above, the processor unit 202 outputs a signal indicating responsive action is to be taken in response to a physiological parameter of the user not having a desired value. In cases where the input device 116 comprises different types of physiological data sensors (i.e. sensors configured to collect different types of physiological data), the processor unit 202 may be configured to output the signal indicating responsive action is to be taken in response to a combination of two or more physiological parameters not having a desired value. The combination of physiological parameters required to trigger an output signal may be predetermined.

The processor unit 202 may output a single type of signal in response to detecting that a physiological parameter does not have a working value (i.e. a signal indicating a single responsive action is to be taken). Alternatively, the processor unit 202 may output a set of signals each indicating a respective responsive action is to be taken. The set of signals may comprise any combination of: 1) the signal to brake the actuators 140-146; 2) the signal to suspend operation of the end effector or surgical instrument 138; 3) the alert signal to the audio output device 108; and 4) the alert signal to the visual display device 112.

In the examples described above, sensors 314_{A,B} were described as physiological data sensors arranged to collect physiological data from the user of input device 116, and the processor unit 202 was arranged to generate an output signal in response to determining from the collected data that at least one physiological parameter of the user did not have a desired value. In another set of examples, the user input device 116 comprises sensors configured to collect data associated with the user's use of the input device. That is, the input device 116 may comprise sensors that collect data that characterises the user's use of the input device 116. Put another way, the sensors may collect data that characterises the user's manipulation of the input device 116 in some way. The processor unit 202 may then generate an output signal indicating a responsive action is to be taken in response to determining from the collected data that a parameter characterising the user's control of the user input device 116 does not have a desired value.

For example, sensors 314_{A,B,C} may instead be touch sensors configured to sense the user's touch. In other words, each touch sensor may detect whether it is or is not in contact with the user. The touch sensors may be, for example, capacitive sensors. The touch sensors may be spatially positioned on the user input device 116 so that data from the touch sensors is indicative of the user's hand position on the user input device 116 during use. In this example, the parameter characterising the user's use of the input device 116 is the user's hand position on the input device 116.

The touch sensors may comprise a first subset of one or more sensors positioned on the input device 116 to be in contact with the user's hand during normal use of the input device 116 to control operation of the surgical robot 102. ‘Normal use’ may refer to the case when the user's hand is in an intended, or desired position on the input device 116. The intended, or desired, position may be a specified position, e.g. an ergonomic position. In the present example, the first subset of sensors are sensors 314_{A,B}, which are positioned on the grip portion 308 of the controller 302. Thus, sensors 314_{A,B} are positioned so that, when the user grips the controller 302 at the grip portion 308, the user's hand is in contact with sensors 314_{A,B}.

The touch sensors may additionally comprise a second subset of one or more sensors positioned on the input device 116 to not be in contact with the user's hand during normal use of the input device 116. In other words, the second subset of one or more sensors are positioned so that, when the user's hand is in the intended position on the input device 116, the hand is not in contact with any of the second subset of one or more sensors. Contact between the user's hand at least one of the second subset of sensors therefore indicates the user's hand is not in the intended, or desired, position on the input device 116. Continuing the present example, the second subset of one or more sensors includes sensor 314_{C}. Sensor 314_{C} is positioned on the head portion 310 of the controller 302. Thus, when the user grips the controller 302 at the grip portion 308, the user's hand is not in contact with sensor 314_{C}.

The touch sensors might include both the first and second subset of sensors; i.e. sensors indicating both a correct and incorrect position for the user's hands on the user input device 116. Alternatively, the touch sensors might include only one of the first and second subsets of sensors, i.e. only the first subset or only the second subset.

Data collected from the first and/or second subset of sensors is communicated to the processor unit 202 over data path 206 of communication link 118. The processor unit 202 can operate to analyse the collected data received from the touch sensors to determine whether the user's hand is in the intended, or desired, or correct, position on the user input device 116. Because the data from the touch sensors is indicative of the user's hand position on the user input device, the processor unit 202 can analyse the data to determine whether the parameter associated with the user's control of the user input device (in this example, the user's hand position on the input device 116) has a desired working value (e.g. the user's hand being in the intended position). To do this, the processor unit 202 might access a set of prestored relationships between sensor data values for sensors 314_{A,B,C} and hand positions on the input device 116. These set of relationships can be stored in memory 204. The relationships might define a set of associations between sensor data values and classifications of hand positions, e.g. correct, or desired, hand positions and incorrect, or undesired, hand positions. Memory 204 may store associations between a first set of senor data values with a set of one or more desired or intended hand positions, and/or a second set of sensor data values with a set of one or more undesired hand positions. In the current example, the first set of sensor data values may be values output by sensors 314_{A,B} when in contact with the user's hand. The second set of sensor data values may be values output by sensors 314_{C} when in contact with the user's hand, and/or values output by sensors 314_{A,B} when not in contact with the user's hand.

Thus, in summary, the processor unit 202 can analyse the data collected from touch sensors 314_{A,B,C} to determine whether the user's hand is in a desired position on the user input device 116 by:
comparing the sensed data to data values stored in memory 204 that are associated with a set of one or more correct hand positions on user input device 116 and/or data values associated with a set of one or more incorrect hand positions on user input device 116; and
determining whether the user's hand is in a correct or incorrect position in dependence on the comparison.

If the processor unit 202 determines from the data collected from touch sensors 314_{A,B,C} that the user's hand is in a correct position on the input device 116, no further action may be taken. In contrast, if the processor unit 202 determines from the collected data that the user's hand is not in a correct position, the processor unit 202 outputs a signal indicating responsive action is to be taken. This signal may be a feedback signal that causes a component of the robotic system 100 to indicate to the user (via means of sensory feedback) that responsive action is to be taken. Various examples of this will now be described.

In one example, the processor unit 202 outputs a feedback signal to audio output device 108 and/or the visual display unit 112 that causes audio and/or visual feedback to be provided to the user that indicates the user's hand is in an incorrect position. For example, the audio output device 108 might, in response to receiving a feedback signal from processor unit 202, output an audio signal indicating the user's hand is in an incorrect position. In some examples, the audio signal might convey adjustments that are to be made to the user's sensed hand position to bring it into a correct position. The determination of what adjustments are needed to the user's hand position may be made by the processor unit 202 from the data collected from sensors 314_{A,B,C}. An indication of these adjustments may be included within the feedback signal output from the processor unit 202.

The visual display device 112 might, in response to receiving a feedback signal from processor unit 202, output a visual display indicating the user's hand is in an incorrect position. The visual display might contain a notice that the user's hand is in an incorrect position. Alternatively, or in addition, the visual display might include an illustration of a correct position of the hand and/or adjustments to be made to the user's sensed hand position to bring it into a correct position. The determination of what adjustments are needed to the user's hand position may be made by the processor unit 202 from the data collected from sensors 314_{A,B,C}. An indication of these adjustments may be included within the feedback signal output from the processor unit 202.

In another example, the processor unit 202 outputs a feedback signal to the user input device 116. This feedback signal might cause the user input device 116 to provide haptic and/or visual feedback to the user indicating the user's hand is in an incorrect position. For example, the user input device 116 might include one or more actuators to provide force or vibrational feedback to the user (not shown in FIG. 3). The actuator(s) might be located within the controller 302. In one implementation, the actuators might be located under the one or more of the sensors 314_{A,B,C}. The actuator(s) might for example be located under sensor 314_{C}. This can conveniently enable a user to receive direct haptic feedback if their hand is in the incorrect position grasping the head portion of the controller 302. Alternatively, the actuator(s) might be located under sensors 314_{A,B}. In this way, the haptic feedback can guide the user's hand to the correct placement on the controller 302.

The user input device 116 might include one or more light output devices (not shown in FIG. 3) to provide visual feedback to the user indicating the user's hand is in an incorrect position. For example, controller 302 may include one or more panels each arranged to be illuminated by one or more light output devices. The light output devices may therefore be mounted beneath the panels. The panels might be included within portions of the controller 302 in contact with the user's hand when in the correct position and/or included within portions of the controller 302 not in contact with the user's hand when in the correct position. In other words, the panels might indicate a correct and/or incorrect position of the user's hands. The feedback signal from processor 302 might cause the panels within portions of the controller in contact with the user's hands when in the correct position to be illuminated and/or the portions of the controller not in contact with the user's hands when in the correct position to be illuminated. If both types of panels are to be illuminated, they may be illuminated in different colours. Illuminating the panels in portions of the controller 302 not in contact with the user's hands when in the correct position indicates to the user they are holding the user input device incorrectly. Illuminating the panels in portions of the controller 302 in contact with the user's hands when in the correct position serves as a visual guide to the user to adjust their hand position.

The processor unit 202 may output a single type of feedback signal in response to detecting from the collected data that the user's hand is not in a desired position. Alternatively, the processor unit 202 may output a set of feedback signals. The set of signals may comprise any combination of: 1) the feedback signal to audio output device 108; 2) the feedback signal to visual display device 112; 3) the feedback signal to the user input device 116 to cause the user input device to provide haptic and/or visual feedback to the user.

In the above-described example, the set of sensors that collected data to characterise the user's use of input device 116 were touch sensors, and the parameter characterising the user's use of the input device 116 was the user's hand position on the input device 116. In another set of examples, the parameter characterising the user's use or manipulation of the input device 116 may relate to the movement of the user input device 116.

For example, the parameter might include: (i) the range of motion through which the input device 116 is manipulated and/or (ii) the force applied by the user to the input device 116 and/or (iii) the orientation of the user input device 116 during use by the user and/or (iv) the frequency components of movements of the controller 302. Each of these parameters may have a desired, or acceptable range of working values. These ranges of working values may be representative of a generally safe operation of the robotic arm. For example, an extreme range of motion of the input device may correspond to an extreme range of motion of the surgical instrument unlikely to be required in a typical surgical procedure. Similarly, applying an excessive force to the input device 116 might inadvertently cause a large force to be applied by the surgical instrument to the patient. The frequency components of the hand controller movements may also have a desired range, indicating movements of the hand controller 302 resulting from natural tremors of the user's hand when the user is holding or grasping the controller 302. Excessive low frequency components in the controller movement may indicate the user is fatigued, or intoxicated. Excessive high frequency components in the controller movement may indicate unsafe levels of hand shakiness

To monitor parameter (i), data collected from sensors 312_{A,B,C} may be used to calculate the position of the hand controller 302 over time during use and thus calculate the range of motion through which the controller 302 is manipulated during use. The calculated position may be a position in 3-D space. The position of the hand controller 302 may be calculated by a processor internal to the user input device 116 from the data collected by sensors 312_{A,B,C} as described above and communicated to the processor unit 202 within the control unit 104. Alternatively, the position of the hand controller 302 may be calculated by the processor unit 202 from the joint positions of linkage 304 sensed from sensors 312_{A,B,C}. Either way, signals indicating the position of the controller 302 are communicated from the device 116 to the processor unit 202. These signals may contain position data for the controller 302 (if the position is calculated by the user input device 116) or joint position data for the joints of linkage 304 (if the position of controller 302 is calculated by the processor 202).

The processor unit 202 may then process the received data indicating the positions of the hand controller 302 to monitor the range of motion through which the hand controller is moved to detect whether the hand controller 302 has been moved through a range of motion that exceeds a specified working range. The working range of motion may be specified in terms of end-of-range positions. In other words, the working range of motion of the hand controller 302 may define a 3-D working volume in space through which the controller 302 can be moved. If the controller 302 is calculated from the sensed data to be at a spatial position within the working volume, then the processor determines that the controller has not exceeded its working range of motion. If the controller 302 is calculated to be at a spatial position outside the working volume, then the processor determines that the controller 302 has exceeded its working range of motion. Alternatively, the working range of motion may specify a maximum magnitude of distance through which the controller 302 can be moved in one motion. This may be defined by specifying the maximum magnitude of distance through which the controller 302 can be moved within a specified time interval. In this case, the processor unit 202 may analyse the received position data of the controller 302 to monitor the distance the controller 302 is moved through over time to determine whether there is a time interval in which the controller is moved a distance that exceeds the specified maximum magnitude. In response to identifying such a time interval, the processor unit 202 determines that the controller 302 has exceeded its working range of motion. If the processor unit 202 cannot identify such a time interval, it determines that the controller 302 has not exceeded its working range of motion.

To monitor parameter (ii), the user input device 116 may be equipped with one or more torque sensors for measuring the torque applied about respective one or more joints of the articulated linkage 304. FIG. 3 shows example torque sensors 316_{A,B,C}. Each torque sensor measures the torque applied about a respective joint of the linkage 304, e.g. during manipulation or use of the controller 302 by the user. The sensed torque data collected by sensors 316_{A,B,C} can then be communicated in a data signal to the processor unit 202 of control unit 104 over data link 118. The processor unit 202 can analyse the sensed torque data received from sensors 316 to determine whether the force applied by the user on the controller 302 exceeds a maximum working value, or specified threshold. The processor unit 302 may determine whether the user-applied force has exceeded the specified threshold by determining whether the sensed torque from sensors 316_{A,B,C} exceeds a specified threshold. This may be done using a number of conditions, for example: 1) by analysing the received sensed data from the torque sensors 316_{A,B,C} to determine whether the torque sensed by any one of the sensors exceeds a specified threshold; 2) by analysing the received sensed data from the torque sensors 316_{A,B,C} to determine whether the average torque sensed by the sensors 316_{A,B,C} exceeds a specified threshold; and 3) by analysing the received sensed data from the torque sensors 316_{A,B,C} to determine whether the total torque sensed by sensors 316_{A,B,C} exceeds a specified threshold. The processor unit 202 may determine whether the measured torque exceeds a specified threshold using one of conditions 1) to 3); or alternatively using a combination of two of conditions 1) to 3) (e.g. condition 1) and 2); condition 1) and 3) or condition 2) and 3)) or using all three conditions. If the processor unit 202 determines from one or more of conditions 1) to 3) as appropriate that the sensed torque has exceeded a specified threshold, it determines that the force applied by the user on the controller 302 has exceeded a specified threshold. This is based on the assumption that the sensed torque in sensors 316 results from the force applied by the user on the controller 302. If the processor unit 202 determines from one or more of conditions 1) to 3) as appropriate that the torque measured by sensors 316_{A,B,C} does not exceed a specified threshold, it determines that the force applied by the user on the controller 302 does not exceed the specified threshold.

Parameter (ii) may alternatively be monitored using one or more accelerometers (not shown in FIG. 3) housed within the controller 302. The accelerometers may be fast with the body of the controller 302. The or each accelerometer may be arranged to measure acceleration along one or more axes. If the accelerometers are fast with the body of the controller 302, the accelerometers can measure the acceleration of the controller 302 along one or more axes, and thus the sensed data from the accelerometers provides an indication of the force applied to the controller 302. The sensed data can be provided to the processor unit 202 along the data link 118. The processor unit 202 can analyse the sensed data from the accelerometers to determine whether the forced applied to the controller 302 exceeds a specified threshold. This may be force applied along one or more directional axes, or the magnitude of the force applied to the controller.

To monitor parameter (iii), data collected from the linkage sensors (e.g. gimbal sensors) may be used to calculate the orientation of the hand controller 302 over time during use. The calculated orientation may be an orientation in 3-D space. The orientation of the hand controller 302 may be calculated by a processor internal to the user input device 116 from the data collected by the sensors as described above and communicated to the processor unit 202 within the control unit 104, or alternatively be calculated by processor unit 202 from the collected data from the sensors. The processor unit 202 may then process the received data indicating the orientation of the hand controller 302 to detect whether the orientation of the controller is within a specified range of working values. The specified range of working values may define a range of acceptable orientations for the controller 302. The range of acceptable orientations may be specified relative to one, two or three axes.

To monitor parameter (iv), data collected from sensors 312_{A,B,C} may be used to calculate position data indicating the position of the hand controller 302 over time during use. The position of the hand controller may be calculated from the joint positions of the linkage 304 sensed from sensors 312_{A,B,C} by a processor internal to the user input device 116. Alternatively, the position of the hand controller 302 may be calculated by the processor unit 202 from the joint positions of linkage 304 sensed from sensors 312_{A,B,C}. Either way, signals indicating the position of the controller 302 are communicated from the device 116 to the processor unit 202. These signals may contain position data for the controller 302 (if the position is calculated by the user input device 116) or joint position data for the joints of linkage 304 (if the position of controller 302 is calculated by the processor 202). The processor unit 202 may therefore track the position of the hand controller 302 in space over time using the signals received from the user input device 116.

The processor unit 202 may perform a frequency analysis (e.g. a Fourier analysis) of the position data for the controller 302 to determine the frequency components of the movements of the controller 302. That is, the processor unit 202 can perform the frequency analysis of the position data to represent movements of the controller 302 over time (i.e. in the temporal domain) as a combination of different frequency components. The processor unit 202 may then determine whether the frequency components of the controller movements are within an acceptable working range. The working range may define a band of acceptable component frequencies. For example, component frequencies below the lower limit of the band may indicate fatigue or intoxication. Component frequencies above an upper limit of the band may indicate unsteadiness (e.g. shakiness, or tremoring). If the frequency-analysed position data for the controller 302 contains an amount of low-frequency components outside the working frequency band that exceeds a specified threshold (defined in terms of maximum amplitude or number of discrete frequency components), or contains an amount of high-frequency components outside the working frequency band that exceeds a specified threshold (defined in terms of maximum amplitude or number of discrete components), then the processor 202 may determine that the frequency components of the hand controller movements are not within an acceptable working range. It has been appreciated that analysing the hand controller movements in the frequency domain can make anomalies in the user's movement more discernible than they would be in the temporal domain. For example, low frequency components of the controller movement (which might be caused by fatigue or intoxication) might not be visible from the controller's position data in the time domain but would be apparent in the frequency domain. As another example, if the controller is being manipulated through a complex pattern of movements, high frequency components of those movements might not be visible from the position data in the time domain but would be apparent in the frequency domain.

The processor unit 202 may alternatively perform the frequency analysis on data sensed by torque sensors 316_{A,B,C} over time or on the data sensed by the accelerometer (if present) over time.

The desired values, or working values, or ranges, associated with parameters (i) to (iv) related to the movement of the user input device 116 may be stored in memory 204 to be accessed by the processor unit 202.

If the processor unit 202 determines that one of the parameters (i) to (iv) being measured does not have a desired working value (i.e. a value within an acceptable working range), it generates and outputs a signal indicating responsive action is to be taken. This signal may be output to another component of the robotic system 100 to cause that component to perform a dedicated responsive action. Various examples of this will now be described.

In response to detecting that parameter (i) does not have a desired working value, the processor unit 202 may output a feedback signal to audio output device 108 and/or visual display device 112. Audio output device 108 may in response output an audio signal indicating the device 116 has exceeded its desired range of motion to provide audio feedback to the user. The visual display device 112 may display an image indicating the device 116 has exceeded its desired working range of motion to provide visual feedback to the user. The image could be pictorial, or a written message. Alternatively or in addition, the processor unit 202 may output a haptic feedback signal to the user input device 116 that provides haptic feedback to the user if the user manipulates the controller 302 in a way that exceeds the desired range of motion. That feedback could be in the form of vibrations, or increased resistance to movement of the controller 302 that further exceeds the desired range of motion.

In response to detecting that parameter (ii) does not have a desired working value, the processor unit 202 may output a feedback signal to audio output device 108 and/or visual display device 112. Audio output device 108 may in response output an audio signal indicating the force applied to device 116 has exceeded a predetermined value to provide audio feedback to the user. The visual display device 112 may display an image indicating the force applied to device 116 has exceeded a predetermined value to provide visual feedback to the user. The image could be pictorial, or a written message. Alternatively, or in addition, the processor unit 202 may output a haptic feedback signal to the user input device 116 that provides feedback to the user. That feedback could be in the form of vibrations of the controller 302.

In response to detecting that parameter (iii) does not have a desired working value, the processor unit 202 may output a feedback signal to audio output device 108 and/or visual display device 112. Audio output device 108 may in response output an audio signal indicating the device 116 is not in a desired working orientation to provide audio feedback to the user. The visual display device 112 may display an image indicating the device 116 is not in a desired working orientation to provide visual feedback to the user. The image could be pictorial, or a written message. Alternatively or in addition, the processor unit 202 may output a haptic feedback signal to the user input device 116 that provides haptic feedback to the user. That feedback could be in the form of vibrations, or increased resistance to movement of the controller 302 that further orientates the controller outside its working range of orientations.

In response to detecting that parameter (iv) does not have a desired working value, the processor unit 202 may output a feedback signal to audio output device 108 and/or visual display device 112. Audio output device 108 may in response output an audio signal indicating the frequency of oscillations of the controller 302 are not within a desired frequency range to provide audio feedback to the user. The visual display device 112 may display an image indicating the frequency of oscillations of the controller 302 are not within a desired frequency range to provide visual feedback to the user. The image could be pictorial, or a written message. Alternatively, or in addition, the processor unit 202 may output a braking signal to brake the actuators 140-146 as described above. This may ensure the robot arm is not controlled by a user who is not be in a suitable physiological state.

The above description describes various examples of how sensors located on the user input device 116 can be used to non-obtrusively collect data to determine whether a parameter associated with the user's operation of the surgical robot has a desired working value. Examples will now be described for alternative approaches for non-obtrusively collecting data relating to the user's control of the surgical robot using other sensory devices of the user console 166.

In one such set of examples, the image capture device 158 captures images of the user during use, i.e. as the user controls the surgical robot 102 through manipulation of the user input device 116. The captured images are then communicated to processor unit 202 through data link 160. The processor unit 202 may then perform image analysis on the captured images to monitor one or more physiological parameters of the user. For example, the processor unit 202 may perform the image analysis to determine the heart rate or breathing rate of the user. The breathing rate may be determined from movements of the user's chest identified from analysing a sequence of the captured images from image capture device 158. Heart rate may be determined by analysing a sequence of captured images to detect facial skin colour variation caused by blood circulation. The skin colour variation may be detected using image processing techniques including independent component analysis (ICA), principle component analysis (PCA) and fast Fourier transform (FFT). As another example, the processor unit 202 may analyse the captured images to detect the pupillary response of the user (i.e. the extent to which the user's pupils are dilated or constricted).

The processor unit 202 may then determine if the values of the physiological parameters are acceptable working values, e.g. within an acceptable working range. For example, the processor unit 202 may determine whether the user's breathing and/or heart rate is above a minimum level (indicating full consciousness) and below a maximum level (possibly indicating undesirable high levels of stress); and/or whether the level of dilation of the user's pupils is above a minimum threshold (potentially indicating suitable levels of engagement) and below a maximum threshold (potentially indicating undesirably high adrenaline levels, or the effects of intoxication through drugs). The processor unit 202 may determine if the values of the physiological parameters have a desired value using stored values for the parameters in memory 204.

In response to detecting that a user's physiological parameter does not have a desired value, the processor unit 202 generates and outputs a feedback signal indicating responsive action is to be taken. That signal may be any one of the signal types described above, e.g. a braking signal to brake the actuators 140-146, or a feedback signal to audio output device 108 and/or image display device 112, or a haptic feedback signal to the user input device 116.

In another set of examples, the audio capture device 162 captures audio data (e.g. sounds emitted from the user) and communicates an audio signal indicating the captured sounds to the processor 202 by data link 164. The processor 202 unit may perform audio analysis on the captured sounds to monitor the state of the user. For example, the processor unit 202 may perform speech analysis on the captured sounds to identify words or phrases spoken by the user. This may be done to identify certain words or phrases that indicate responsive action might need to be taken. For example, a swear word, or multiple swear words, may indicate that the user has made an error during the surgical procedure. As another example, a phrase may be used to indicate that the user requires assistance, for example by indicating that the user is fatigued, or not feeling well. In other words, the processor unit 202 might perform speech analysis on the audio data captured by the audio capture device 162 to determine whether one of a set of specified words and/or phrases has been spoken by the user that indicate responsive action is to be taken. Alternatively, the processor unit 202 may perform speech analysis on the captured audio data to classify the tone of voice of the user according to a set of specified tones. The specified set of tones might include, for example, calm, worried, panicked, stressed, angry etc.

If the processor unit 202 identifies from the analysed audio data that the user has spoken one of the specified words or phrases, or the determines from the analysis that the user's tone of voice is one of the specified tones, it generates and outputs a feedback signal indicating responsive action is to be taken. The feedback signal may be any of the feedback signals described above.

In another set of examples, the user console 166 may comprise a breathalyser (not shown in FIG. 1) to analyse the user's breath to detect alcohol levels. The user may be required to breathe into the breathalyser before beginning a procedure, i.e. before the user input device 116 can be used to manipulate the robot arm. For example, the robotic system may be configured to operate in a locked mode and an active mode. In locked mode, movement of the user input device 116 causes no corresponding movement of the robot arm or end effector. In active mode, movement of the input device 116 causes corresponding movement of the robot arm to move the end effector to a desired position and orientation as described above. The processor unit 202 may be configured to receive a signal from the breathalyser indicating the alcohol levels in the user's blood when the robotic system is in locked mode. The processor unit 202 may then analyse the received signal to determine whether the alcohol level is below a specified threshold. In response to determining that it is, the processor unit may output a signal to the user input device 116 and robot arm that transitions the operational mode from locked to active. If the processor unit 202 determines that the alcohol level exceeds the threshold, it causes the robotic system to remain in locked mode.

In an alternative arrangement, the processor 202 may receive a signal from the breathalyser indicating the user's alcohol level when the robotic system is in active mode. If the processor unit determines the alcohol level exceeds the specified threshold, it outputs a signal causing the robotic system to transition to the locked mode.

The robotic system 100 may optionally comprise a data logger 168 for logging the data collected from the user (e.g. from the sensors on the user input device 116 and/or from the image capture device 158 and audio capture device 162). The datalogger 168 may additionally log the activity of the processor unit 202 over time, for example by logging: (i) each time the processor unit outputs a feedback signal; and (ii) the physiological parameter determined to have a value outside its working range that caused that feedback signal to be emitted. The datalogger may log additional data, such as the time each feedback signal was emitted.

The datalogger is shown in FIG. 1 as being coupled to the control unit 104, but this is merely an example arrangement. In other arrangements the datalogger 168 may be directly connected to the sensors of the user input device 116 and/or the image capture device 158 and the audio capture device 162.

The datalogger 168 may be configured to identify, or characterise, stages/steps of the surgical procedure being performed from the data collected from the sensors on the user input device 116. For example, data collected over multiple procedures from the sensors on the user input device (e.g. the position data of the joints of the linkage 304 and/or the torque applied about each joint of the linkage 304) may be analysed offline and used to characterise one or each of a number of surgical procedures as a number of discrete steps, or stages. Having characterised the surgical procedure, the datalogger 168 may be configured to use the data collected from the user and the data collected from the processing unit 102 to associate the feedback signals to steps of the surgical procedure. This may enable patterns in the user's behaviour to be identified and associated with steps of the surgical procedure, which might be useful in identifying training or other development needs.

For example, the datalogger may be able to determine one or more of the following:

(i) the step of the surgical procedure in which the user is most likely to adopt a certain physiological state, such as fatigue or stress;
(ii) the time since the beginning of the procedure the user is user is most likely to adopt a certain physiological state, such as fatigue or stress;
(iii) the step of the procedure for which it is most likely that an error will occur (e.g. determined from the step for which it is most likely that a feedback signal is communicated from the processor unit 202).

The datalogger may also be able to identify markers, or targets, for the surgical procedure to maintain suitable performance levels, for example:

(i) the datalogger may determine that the likelihood of an error occurring exceeds a specified threshold if the procedure is not completed within a specified amount of time of the procedure starting;
(ii) the datalogger may determine that the likelihood of an error occurring exceeds a specified threshold if a particular stage of the surgical procedure is not reached, or not completed, within a specified time of the procedure starting.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A surgical robotic system, comprising:
a surgical robot;
a user input device coupled to the surgical robot and manipulatable by a user to control operation of the surgical robot, the user input device comprising one or more sensors configured to collect data as the user manipulates the user input device; and
a processor unit configured to:
analyse the collected data to determine whether a parameter associated with the operation by the user of the surgical robot has a desired working value, the determination comprising analysing the collected data to calculate a time-averaged value of the parameter over a specified period of time, and determining whether the time-averaged value of the parameter is within a specified target range; and
generate an output signal indicating responsive action is to be taken in response to determining from the collected data that the time-averaged value of the parameter is not within the target range;
wherein the parameter associated with the operation by the user of the surgical robot is one or more of:
a physiological parameter of the user, the one or more sensors being configured to collect physiological data for the user; and
indicative of the user's manipulation of the user input device.

2. A surgical robotic system as claimed in claim 1, wherein the one or more sensors comprise a set of one or more sensors positioned on the user input device to be in contact with the user's hands as the user manipulates the user input device to control operation of the surgical robot.

3. A surgical robotic system as claimed in claim 1, wherein the processor unit is configured to analyse the collected data to determine whether the physiological parameter for the user has a value within a specified range; and to generate the output signal in response to determining from the collected data that the parameter does not have a value within the specified range.

4. A surgical robotic system as claimed in claim 1, wherein the processor unit is configured to determine whether a combination of two or more physiological parameters have desired working values, and to generate the output signal in response to determining that the two or more physiological parameters do not have desired working values.

5. A surgical robotic system as claimed in claim 1, wherein the set of one or more sensors are configured to measure one or more of: temperature, pulse rate; blood oxygen saturation level; perspiration rate; ionic concentration in perspiration; hydration level; blood pressure; hand steadiness.

6. A surgical robotic system as claimed in claim 1, wherein the surgical robot comprises a plurality of limbs interconnected by joints; and a set of actuators configured to drive the joints, and the output signal is a braking signal to brake the actuators.

7. A surgical robotic system as claimed in claim 1, wherein the surgical robot comprises a surgical instrument, and the output signal causes the instrument to be disabled.

8. A surgical robotic system as claimed in claim 1, wherein the surgical robotic system comprises a speaker coupled to the processor unit, the speaker being configured to output an audio alert signal in response to receiving the output signal from the processor unit.

9. A surgical robotic system as claimed in claim 1, wherein the surgical robotic system comprises a visual display coupled to the processor unit, the visual display being configured to output a visual alert signal in response to receiving the output signal from the processor unit.

10. A surgical robotic system as claimed in claim 1, wherein the parameter associated with the operation by the user of the surgical robot relates to the user's interaction with the user input device when manipulating the input device to control operation of the surgical robot.

11. A surgical robotic system as claimed in claim 1, wherein the parameter is indicative of one or more of:
the force applied by the user to the user input device when manipulating the input device to control operation of the surgical robot;
a range of motion through which the user manipulates the user input device;
an orientation of the user input device when manipulated by the user to control operation of the surgical robot; and
the frequency components of movements of a hand controller of the user input device when held by the user.

12. A surgical robotic system as claimed in claim 11, wherein the one or more sensors are configured to collect data indicative of the position of the hand controller over time, and the processor is configured to analyse the collected data by performing a frequency analysis on the collected data to determine the frequency components of the movements of the hand controller when held by the user.

13. A surgical robotic system as claimed in claim 1, wherein the output signal is haptic feedback signal, the processor unit configured to communicate the haptic feedback signal to the user input device.

14. A surgical robotic system as claimed in claim 10, wherein the parameter is the user's handgrip on the user input device, the processor unit being configured to analyse the collected data to determine whether the user's handgrip is in a specified desired position.

15. A surgical robotic system as claimed in claim 14, wherein the output signal generated by the processor unit is a feedback signal indicative of the specified desired position.

16. A surgical robotic system as claimed in claim 1, wherein the user input device comprises one or more light output devices to provide visual feedback to a user.

17. A surgical robotic system as claimed in claim 1, wherein the parameter is indicative of frequency components of data sensed by torque sensors or data sensed by one or more accelerometer.

18. A surgical robotic system as claimed in claim 15, wherein the output signal is a visual feedback signal, the processor unit being configured to communicate the visual feedback signal to a visual display unit to cause the visual display unit to display the specified desired position of the user's handgrip.

19. A surgical robotic system as claimed in claim 1, further comprising a datalogger for logging data collected from the one or more sensors during a surgical procedure performed by means of the surgical robot.

20. A method of monitoring performance during manipulation of a user input device of a surgical robotic system, the surgical robotic system comprising a surgical robot and a user input device coupled to the surgical robot and manipulatable by a user to control operation of the surgical robot, the user input device comprising one or more sensors configured to collect data as the user manipulates the user input device, the method comprising:
analysing data collected by the user input device to determine whether a parameter associated with the operation by the user of the surgical robot has a desired working value, the determination comprising:
analysing the collected data to calculate a time-averaged value of the parameter over a specified period of time, and
determining whether the time-averaged value of the parameter is within a specified target range; and
generating an output signal indicating responsive action is to be taken in response to determining from the collected data that the time-averaged value of the parameter is not within the target range;
wherein the parameter associated with the operation by the user of the surgical robot is one or more of:
a physiological parameter of the user, and one or more sensors of the user input device are configured to collect physiological data for the user; and
indicative of the user's manipulation of the user input device.
